(19) United States
(12) Patent Application Publication
(10) Pub. No.: US 2026/0199085 A1
(43) Pub. Date: Jul. 16, 2026

(54) IMPLANTABLE MEDICAL DEVICE

(71) Applicant: Edwards Lifesciences Corporation, Irvine, CA (US)

(72) Inventors: Yong Zhao, Lake Forest, CA (US); Biswa P. Das, Irvine, CA (US)

(21) Appl. No.: 19/563,897

(22) Filed: Mar. 11, 2026

Related U.S. Application Data
(63) Continuation of application No. PCT/US2024/046572, filed on Sep. 13, 2024.
(60) Provisional application No. 63/582,810, filed on Sep. 14, 2023.

(51) Int. Cl. A61F 2/24

(52) U.S. Cl. CPC A61F 2/2418; A61F 2220/005

(57) ABSTRACT

Disclosed herein is an implantable prosthetic valve comprising: an annular frame comprising a plurality of struts; a valvular structure comprising one or more leaflets, each having an inner surface and an outer surface and positioned at the inner surface of the annular frame; wherein at least a portion of an edge portion of the one or more leaflets is suturelessly attached to at least a portion of the plurality of struts of the annular frame with an adhesive composition to form a coupling portion, wherein the coupling portion encapsulates the at least a portion of the plurality of struts and seamlessly extends into the at least a portion of an edge portion of the one or more leaflets. The methods of making the same are also disclosed.

### CROSS-REFERENCE TO RELATED APPLICATION

This application is a continuation of International Application No. PCT/US2024/046572, filed Sep. 13, 2024, which claims the benefit of U.S. Application No. 63/582,810, filed Sep. 14, 2023, the entire disclosures all of which are incorporated by reference for all purposes.

### FIELD

The present disclosure relates to implantable expandable prosthetic devices and methods and apparatuses for such prosthetic devices.

### BACKGROUND

The heart can suffer from various valvular diseases or malformations that result in its significant malfunctioning and ultimately require replacing the native heart valve with an artificial one. Human heart valves, which include the aortic, pulmonary, mitral, and tricuspid valves, function essentially as one-way valves operating in synchronization with the pumping heart. The valves allow blood to flow downstream but block blood from flowing upstream. Diseased heart valves exhibit impairments such as narrowing of the valve or regurgitation, which inhibits the valves’ ability to control blood flow. Such impairments reduce the heart’s blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical, tissue-based, or polymer-based heart valve prostheses can be used to replace impaired native heart valves. More recently, substantial effort has been dedicated to developing replacement heart valves that can be delivered with less trauma to the patient than through open-heart surgery. Replacement valves are being designed to be delivered through minimally invasive procedures and even percutaneous procedures. Such replacement valves often include a tissue-based or polymer-based valvular system that is connected to an expandable frame that is then delivered to the native valve’s annulus.

Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 5,411,522 and 6,730,118, 7,393,360, 7,510,575, and 7,993,394, which are incorporated herein by reference, describe collapsible transcatheter heart valves (THVs) that can be percutaneously introduced in a compressed state on a catheter and expanded in the desired position by balloon inflation or by utilization of a self-expanding frame or stent. In yet another example, U.S. Patent Publication Nos. 2014/0277390, 2014/0277422, 2014/0277427, 2015/0328000, and 2019/0328515, which are incorporated herein by reference in their entireties, describe heart valve prostheses for replacing a native mitral valve, including a self-expanding frame with a plurality of anchoring members that are designed be deployed within a body cavity and prevent axial flow of fluid around an exterior of the prosthesis.

The currently existing tissue-based and polymer-based valve prostheses require suturing of the valvular structures (or leaflets) to other valve components. While suturing works well for tissue leaflets, it presents various challenges for polymeric leaflets. Polymer leaflets often tear at the suture stitch locations. Even further, the region of the leaflet nearest to the frame is often subjected to higher stresses and, therefore, more prone to tearing.

Thus, alternative methods are needed to join polymer leaflets to the prosthetic valves without suturing. Also, there is a need for stronger bonding and reinforcement to extend the durability of the device. These and other needs are at least partially satisfied by the present disclosure.

### SUMMARY

Some of the aspects of the present disclosure relate to implantable prosthetic valves. Some aspects disclosed herein are directed to an implantable prosthetic valve comprising: a) an annular frame having an inner surface and an outer surface wherein the frame has an inflow end and an outflow end, and a central longitudinal axis extending from the inflow end to the outflow end, wherein the annular frame comprises a plurality of struts; a valvular structure comprising one or more leaflets, each having an inner surface and an outer surface and positioned at the inner surface of the annular frame; wherein at least a portion of an edge portion of the one or more leaflets is suturelessly attached to at least a portion of the plurality of struts of the annular frame with an adhesive composition to form a coupling portion, wherein the coupling portion encapsulates the at least a portion of the plurality of struts and seamlessly extends into the at least a portion of an edge portion of the one or more leaflets.

In yet further aspects, the one or more leaflets comprise a first polymer material. In such exemplary and unlimiting aspects, the first polymer material can comprise a polyurethane-based composition, polyurea composition, PTFE, silicon rubber, SIBS, or any combination thereof. Yet, in other aspects, the adhesive composition can comprise a composition comprising a second polymer, an oligomer, a monomer, or a combination thereof. In such aspects, if the second polymer is present, it can be the same or different from the first polymer material.

Also disclosed herein are aspects directed to methods of making any of the disclosed herein implantable prosthetic valves. In one aspect, disclosed is a method comprising providing an annular frame having an inner surface and an outer surface wherein the frame has an inflow end and an outflow end, and a central longitudinal axis extending from the inflow end to the outflow end; wherein the annular frame comprises a plurality of struts; aligning at least a portion of an edge portion of one or more leaflets with a portion of the plurality of struts; and dispersing an adhesive composition to form an adhesive composition configured to couple the portion of the plurality of struts with the at least a portion of the edge portion of the one or more leaflets to form a sutureless coupling portion between the one or more leaflets and the plurality of struts of the annular frame, such that the coupling portion encapsulates the at least a portion of the plurality of struts and seamlessly extends into the at least a portion of an edge portion of the one or more leaflets.

Yet in still further aspects, disclosed is a method comprising providing an annular frame having an inner surface and an outer surface wherein the frame has an inflow end and an outflow end, and a central longitudinal axis extending from the inflow end to the outflow end; wherein the annular frame comprises a plurality of struts; and forming a polymeric valvular structure comprising one or more leaflets within the inner surface of the annular frame such that at least a portion of an edge portion of one or more leaflets form a unibody with at least a portion of the plurality of struts. In such exemplary and unlimiting aspects, the polymeric valvular structure can be molded from a polymer composition within the inner surface of the annular frame.

Additional aspects of the disclosure will be set forth, in part, in the detailed description, figures, and claims that follow, and in part, will be derived from the detailed description or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

FIG. 1 is a perspective view of a conventional sutured prosthetic heart valve.

FIG. 2 shows an exemplary valvular structure.

FIG. 3 is a perspective view of an exemplary implantable prosthetic valve according to some aspects of the disclosure.

FIGS. 4-8 show an exemplary frame of the prosthetic heart valve of FIGS. 1 or 3.

FIG. 9 shows an exemplary coupling portion of the leaflet structures in an exemplary valve of FIG. 3.

FIG. 10 shows exemplary method steps of forming an exemplary coupling portion according to some aspects of the disclosure.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### DEFINITIONS

As used in this application and in the claims, the singular forms “a,” “an,” and “the” include the plural forms unless the context clearly dictates otherwise. Thus, for example, reference to a “leaflet” includes aspects having two or more such leaflets unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The term “or” means “and/or.” Recitation of ranges of values is merely intended to serve as a shorthand method of referring individually to each separate value falling within the range unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint and independently of the other endpoint. Unless stated otherwise, the term “about” means within 5% (e.g., within 2% or 1%) of the particular value modified by the term “about.” It is further understood that if the value is disclosed as “about 1,” then the value of “1” is also disclosed.

As used herein, the terms “optional” or “optionally” mean that the subsequently described event or circumstance may or may not occur and that the description includes instances where said event or circumstance occurs and instances where it does not.

Further, the terms “coupled,” “attached,” and “associated” generally mean electrically, electromagnetically, and/or physically (e.g., mechanically or chemically) coupled or linked and do not exclude the presence of intermediate elements between the coupled or associated items.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of exemplary aspects.

Spatially relative terms, such as “beneath,” “below,” “lower,” “above,” “upper,” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free” when used in the context of a composition or component of a composition that is substantially absent but can be present as an impurity along with other components being added to the composition. In such aspects, the term “substantially free” is intended to refer to trace amounts that can be present in the batched components, for example, it can be present in an amount that is less than about 1% by weight, e.g., less than about 0.5% by weight, less than about 0.1% by weight, less than about 0.05% by weight, or less than about 0.01% by weight of the stated material, based on the total weight of the composition. Yet in other aspects, the term “substantially free,” when used in the context of surface roughness, surface smoothness, defects, and the like, is intended to indicate that the described parameters are substantially absent or at least absent to the degree that cannot affect the performance of the described elements.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation. Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### IMPLANTABLE MEDICAL DEVICE

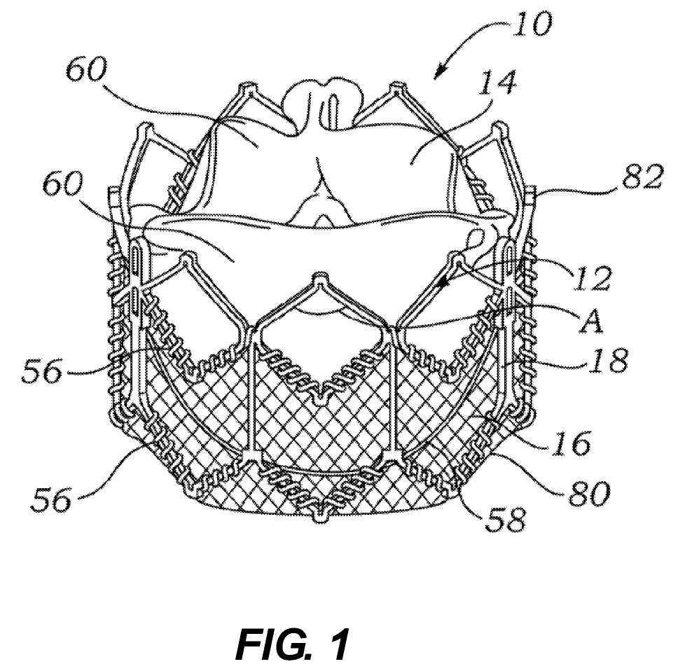

FIG. 1 shows one of the currently used prosthetic valve 10. Valve 10 generally comprises a frame, or stent, 12, a valvular structure 14 supported by the frame, and a skirt 16 secured to the outer surface of the leaflet structure. Valvular structure 14 comprises leaflets 60 positioned together to mimic the natural valvular anatomy of a patient. Valve 10 typically is implanted in the annulus of the native aortic valve but can also be adapted to be implanted in other native valves of the heart or in various other ducts or orifices of the body. Valve 10 has a “lower” end 80 and an “upper” end 82. In the context of the present application, the terms “lower” and “upper” are used interchangeably with the terms “inflow” and “outflow,” respectively. Thus, for example, the lower end 80 of the valve is its inflow end, and the upper end 82 of the valve is its outflow end.

Valve 10 and frame 12 are configured to be radially collapsible to a collapsed or crimped state for introduction into the body on a delivery catheter and radially expandable to an expanded state for implanting the valve at a desired location in the body (e.g., the native aortic valve).

It can be seen that all the elements of the valve 10 are attached with the use of sutures or supporting ports. For example, skirt 16 can be secured to the inside of frame 12 via sutures 56. Leaflet structure 14 can be attached to the skirt via a thin PET reinforcing strip, which enables a secure suturing and protects the pericardial tissue of the leaflet structure from tears. Leaflet structure 14 can be sandwiched between skirt 16 and the thin PET strip. Suture 58, which secures the PET strip and the leaflet structure 14 to skirt 16 can be any suitable suture, such as a polyester suture (e.g., ETHIBOND polyester suture, Johnson & Johnson). Suture 58 desirably tracks the curvature of the bottom edge of leaflet structure 14. Further, in this exemplary valve, leaflets 60 can be secured to one another at their adjacent sides to form commissures 84 of the leaflet structure (the edges where the leaflets come together). Leaflet structure 14 can be secured to frame 12 using suitable techniques and mechanisms. For example, commissures of the leaflet structure are aligned with the support posts 18 and secured thereto using sutures.

Such valve configurations work substantially well for tissue-based (for example, bovine pericardial tissues) leaflets. However, a large number of required sutures and supporting ports is problematic when the valve comprises biocompatible synthetic materials, as it can result in uncontrollable tears, uncontrolled stretching of the suture holes, and, as a result, failure of the valve.

Here disclosed are aspects where the implantable prosthetic device comprises biocompatible polymeric leaflets that are suturelessly attached to the valve frame.

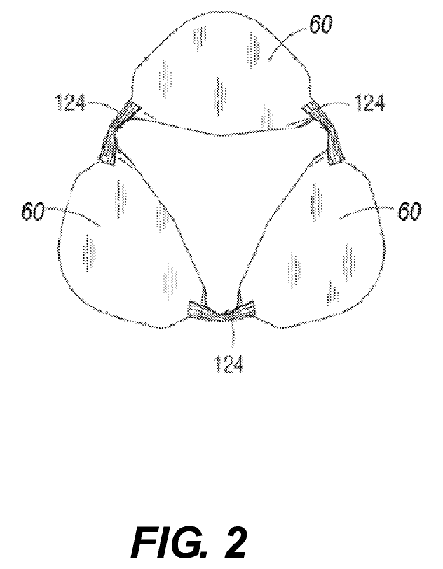

FIG. 2 shows an exemplary leaflet structure. In this exemplary aspect, the valvular structure 14 comprises three flexible leaflets 60 (although a greater or a smaller number of leaflets can be used) that can be arranged to collapse in a tricuspid arrangement. The lower edge of leaflet structure 14 can have an undulating, curved scalloped shape (not shown). By forming the leaflets with this scalloped geometry, stresses on the leaflets are reduced, which in turn improves the durability of the valve. Moreover, by virtue of the scalloped shape, folds, and ripples at the belly of each leaflet (the central region of each leaflet), which can cause early calcification in those areas, can be eliminated or at least minimized. The scalloped geometry also reduces the amount of material used to form the leaflet structure, thereby allowing a smaller, more even crimped profile at the inflow end of the valve. Various configurations of the leaflets in the valve can be found, for example, in U.S. Patent Application Publication No. 2015/0320556 or U.S. Patent No. 8,454,685, the contents of which are incorporated by reference in their entirety.

The leaflets 60 can be secured to one another at their adjacent sides to form commissures of the leaflet structure. A plurality of flexible connectors 124 (as shown in FIG. 2) can be used to interconnect pairs of adjacent sides of the leaflets and to mount the leaflets to the commissure window frame portions 18 (FIG. 1).

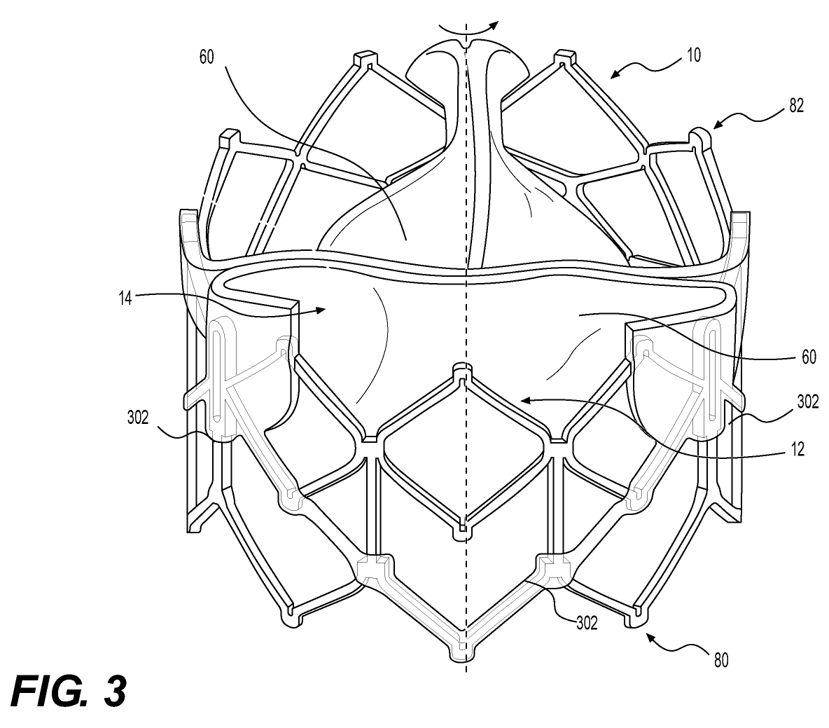

FIG. 3 shows the exemplary implantable prosthetic valve 10 having polymeric leaflets 60 of valvular system 14 attached to frame 12 according to the aspects of the current disclosure.

The annular frame 12, as disclosed herein, has an inner surface and an outer surface (not shown) wherein the frame has an inflow end and an outflow end and a central longitudinal axis extending from the inflow end 80 to the outflow end 82, wherein the annular frame comprises a plurality of struts.

Frame 12 can be made of a plastically-expandable material that permits crimping of the valve to a smaller profile for delivery and expansion of the valve using an expansion device such as the balloon of a balloon catheter. In certain aspects, the expandable material can comprise stainless steel, cobalt-chromium alloy, cobalt-chromium-tungsten-nickel alloy, nickel-cobalt-chromium alloy, nickel-cobalt-chromium-molybdenum alloy, nitinol, polymer, or any combination thereof. In some exemplary and unlimiting aspects, the polymer used to form the frame can comprise polyether ether ketone (PEEK), polytetrafluoroethylene (PTFE), ultra-high-molecular-weight polyethylene (UHMWPE), polyimide, polysulfone (PSU), polyphenylsulfone (PPSU) (filled or unfilled), or any combination thereof. In still further aspects, the polymers used for the frame can comprise radiopaque fillers such as bismuth subcarbonate, bismuth trioxide, barium sulfate, tungsten, and the like.

Exemplary plastically-expandable materials that can be used to form the frame are described below. For example, valve 10 can be a so-called self-expanding valve wherein the frame is made of a self-expanding material such as nitinol. A self-expanding valve can be crimped to a smaller profile and held in the crimped state with a restraining device such as a sheath covering the valve. When the valve is positioned at or near the target site, the restraining device is removed to allow the valve to self-expand to its expanded, functional size.

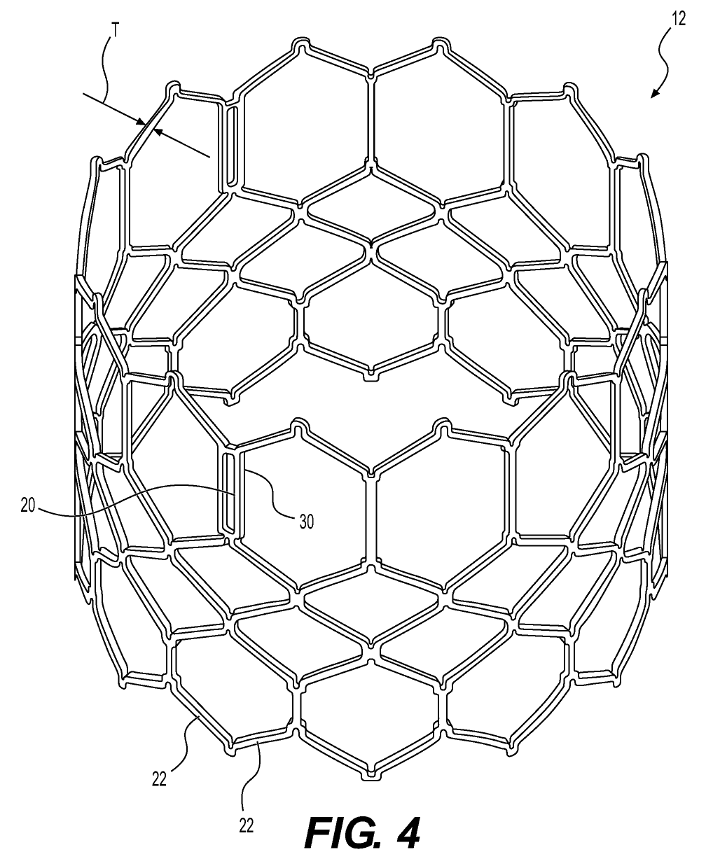

The bare frame 12 is shown in FIG. 4. Frame 12 can be formed with a plurality of circumferentially spaced slots or commissure windows, 20 (three are shown in the illustrated and unlimiting aspect) that are adapted to mount the commissures of the valvular structure 14 to the frame, as described in greater detail below. As discussed above, frame 12 can be made of any of various suitable plastically-expandable materials (e.g., stainless steel, etc.) or self-expanding materials (e.g., nickel-titanium alloy (NiTi), such as nitinol) as known in the art. When constructed of a plastically-expandable material, frame 12 (and thus the prosthetic valve 10) can be crimped to a radially collapsed configuration on a delivery catheter and then expanded at the implantation site by an inflatable balloon or equivalent expansion mechanism. When constructed of a self-expandable material, frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, biocompatible, high-strength alloys (e.g., a cobalt-chromium or nickel-cobalt-chromium alloys), polymers, or combinations thereof. In particular aspects, frame 12 is made of a nickel-cobalt-chromium-molybdenum alloy, such as UNS R30035 alloy (covered by ASTM F562-02, e.g., MP35N® alloy, SPS Technologies, Jenkintown, Pa.). UNS R30035 alloy comprises 35% nickel, 35% cobalt, 20% chromium, and 10% molybdenum by weight. It has been found that the use of UNS R30035 alloy to form frame 12 provides superior structural results over stainless steel. In particular, when UNS R30035 alloy is used as the frame material, less material is needed to achieve the same or better performance in radial and crush force resistance, fatigue resistance, and corrosion resistance. Moreover, since less material is required, the crimped profile of the frame can be reduced, thereby providing a lower-profile prosthetic valve assembly for percutaneous delivery to the treatment location in the body.

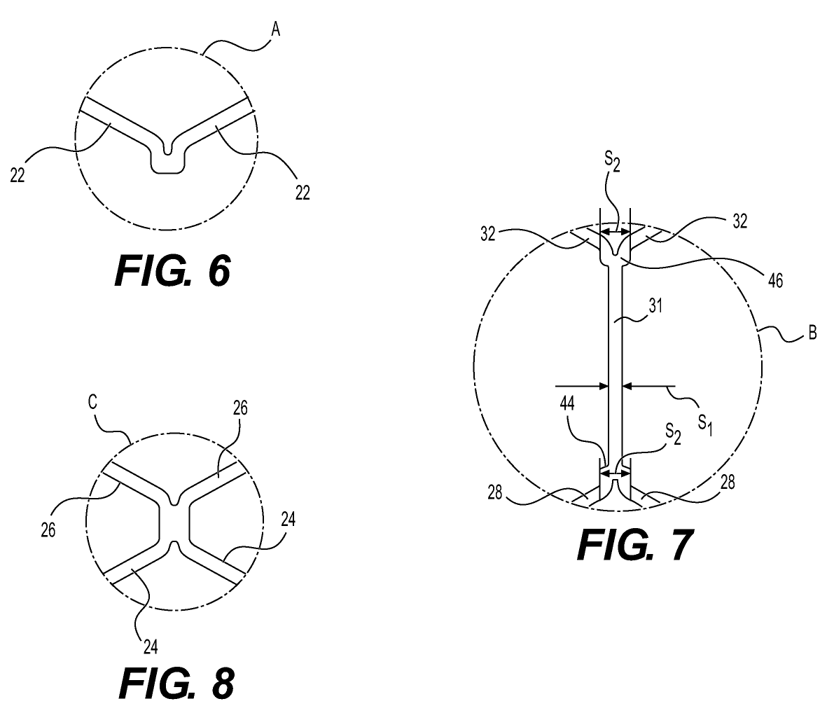

Referring to FIGS. 4-6 and 8, frame 12 in the illustrated aspect comprises a first, lower row I of angled struts 22 arranged end-to-end and extending circumferentially at the inflow end of the frame; a second row II of circumferentially extending angled struts 24; a third row III of circumferentially extending, angled struts 26; a fourth row IV of circumferentially extending angled struts 28; and a fifth row V of circumferentially extending, angled struts 32 at the outflow end of the frame. A plurality of substantially straight axially extending struts 34 can be used to interconnect the struts 22 of the first row I with the struts 24 of the second row II. The fifth row V of angled struts 32 is connected to the fourth row IV of angled struts 28 by a plurality of axially extending window frame portions 30 (which define the commissure windows 20) and a plurality of axially extending struts 31. Each axial strut 31 and each frame portion 30 extends from a location defined by the convergence of the lower ends of two angled struts 32 to another location defined by the convergence of the upper ends of two angled struts 28.

Each commissure window frame portion 30 mounts a respective commissure of the leaflet structure 14. As can be seen, each frame portion 30 is secured at its upper and lower ends to the adjacent rows of struts to provide a robust configuration that enhances fatigue resistance under cyclic loading of the prosthetic valve compared to known, cantilevered struts for supporting the commissures of the leaflet structure. This configuration enables a reduction in the frame wall thickness to achieve a smaller crimped diameter of the prosthetic valve. In certain aspects, the thickness T of frame 12 (FIG. 4) measured between the inner diameter and outer diameter is about 0.48 mm or less.

The struts and frame portions of the frame collectively define a plurality of open cells of the frame. At the inflow end of frame 12, struts 22, struts 24, and struts 34 define a lower row of cells defining openings 36. The second, third, and fourth rows of struts 24, 26, and 28 define two intermediate rows of cells defining openings 38. The fourth and fifth rows of struts 28 and 32, along with frame portions 30 and struts 31, define an upper row of cells defining openings 40. The openings 41 are relatively large and are sized to allow portions of the leaflet structure 14 to protrude, or bulge, into and/or through the openings 40 when the frame 12 is crimped in order to minimize the crimping profile.

As best shown in FIG. 7, the lower end of the strut 31 is connected to two struts 28 at a node or junction 44, and the upper end of the strut 31 is connected to two struts 32 at a node or junction 46. The strut 31 can have a thickness S₁ that is less than the thicknesses S₂ of the junctions 44, 46. The junctions 44, 46, along with junctions 64, prevent the full closure of opening 40. The geometry of the struts 31, and junctions 44, 46, and 64 assists in creating enough space in openings 41 in the collapsed configuration to allow portions of the prosthetic leaflets to protrude or bulge outwardly through openings. This allows the prosthetic valve to be crimped to a relatively smaller diameter than if all of the leaflet material were constrained within the crimped frame.

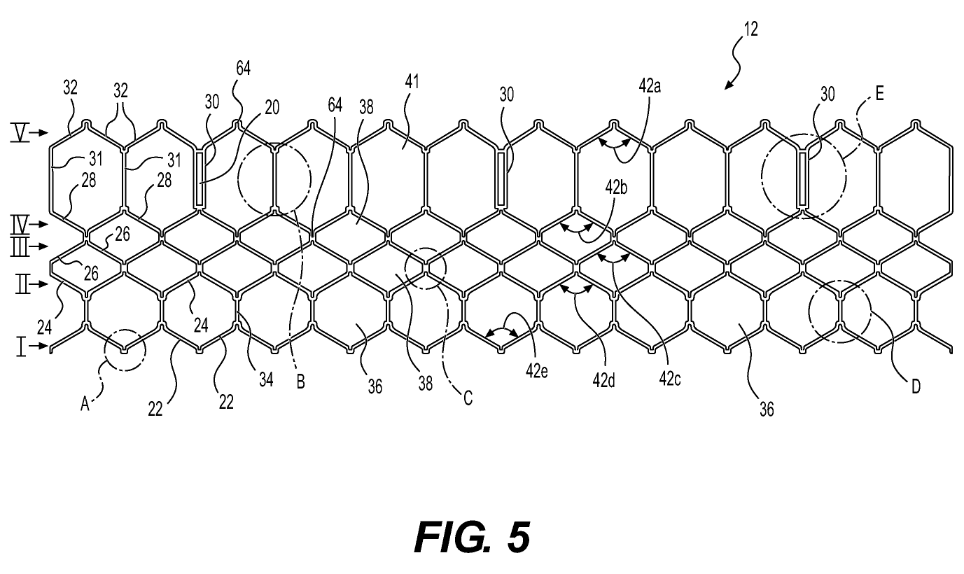

Frame 12 is configured to reduce, prevent, or minimize possible over-expansion of the prosthetic valve at a predetermined balloon pressure, especially at the outflow end portion of the frame, which supports the leaflet structure 14. In one aspect, the frame is configured to have relatively larger angles 42a, 42b, 42c, 42d, 42e between struts, as shown in FIG. 5. The larger the angle, the greater the force required to open (expand) the frame. As such, the angles between the struts of the frame can be selected to limit the radial expansion of the frame at a given opening pressure (e.g., the inflation pressure of the balloon). In some exemplary aspects, these angles are at least 110 degrees or greater when the frame is expanded to its functional size, and even more particularly, these angles are up to about 120 degrees when the frame is expanded to its functional size.

In addition, the inflow 80 and outflow 82 ends of a frame generally tend to over-expand more so than the middle portion of the frame due to the “dog-boning” effect of the balloon used to expand the prosthetic valve. To protect against over-expansion of the leaflet structure 14, the leaflet structure desirably is secured to the frame 12 below the upper row of struts 32. Thus, in the event that the outflow end of the frame is over-expanded, the leaflet structure is positioned at a level below where over-expansion is likely to occur, thereby protecting the leaflet structure from over-expansion.

In some aspects, in a prosthetic valve construction, portions of the leaflets can protrude longitudinally beyond the outflow end of the frame when the prosthetic valve is crimped if the leaflets are mounted too close to the distal end of the frame. If the delivery catheter on which the crimped prosthetic valve is mounted includes a pushing mechanism or stop member that pushes against or abuts the outflow end of the prosthetic valve (for example, to maintain the position of the crimped prosthetic valve on the delivery catheter), the pushing member or stop member can damage the portions of the exposed leaflets that extend beyond the outflow end of the frame. Another benefit of mounting the leaflets at a location spaced away from the outflow end of the frame is that when the prosthetic valve is crimped on a delivery catheter, the outflow end of the frame 12 rather than the leaflets 60 is the proximal-most component of the prosthetic valve 10. As such, if the delivery catheter includes a pushing mechanism or stop member that pushes against or abuts the outflow end of the prosthetic valve, the pushing mechanism or stop member contacts the outflow end of the frame, and not leaflets 40, so as to avoid damage to the leaflets.

Also, as can be seen in FIG. 5, the openings 36 of the lowermost row of openings in the frame are relatively larger than the openings 38 of the two intermediate rows of openings. This allows the frame, when crimped, to assume an overall tapered shape that tapers from a maximum diameter at the outflow end of the prosthetic valve to a minimum diameter at the inflow end of the prosthetic valve.

In certain aspects, the disclosed herein leaflets comprise a first polymer material. Any biocompatible polymers suitable for this desired application can be used. For example and without limitations, biocompatible synthetic materials, or various other suitable natural or synthetic materials are described in U.S. Pat. No. 6,730,118, which is incorporated by reference herein. In still further aspects, the first polymer material can comprise polyurethane-based composition, polyurea composition, polytetrafluoroethylene (PTFE), silicon, styrene isoprene butadiene (SIBS) block copolymers, or any combination thereof. In still further aspects, polyurethane-based composition comprises one or more poly (ether urethanes), poly (ester urethanes), poly (carbonate urethanes), poly (siloxane urethanes), or any combination thereof. In still further aspects, the polyurethane-based composition can comprise polyurethane-polyester composites, polyurethane-polyamide composites, polyurethane-glass fiber composites, polyurethane-carbon fiber composites, or any combination thereof.

In still further aspects, the valvular structure 14 is positioned at the inner surface of the annular frame as shown in FIG. 3. In still further aspects, at least a portion of an edge portion of the one or more leaflets is suturelessly attached to at least a portion of the plurality of struts of the annular frame with an adhesive composition to form a coupling portion 302, wherein the coupling portion 302 encapsulates the at least a portion of the plurality of struts and seamlessly extends into the at least a portion of an edge portion of the one or more leaflets.

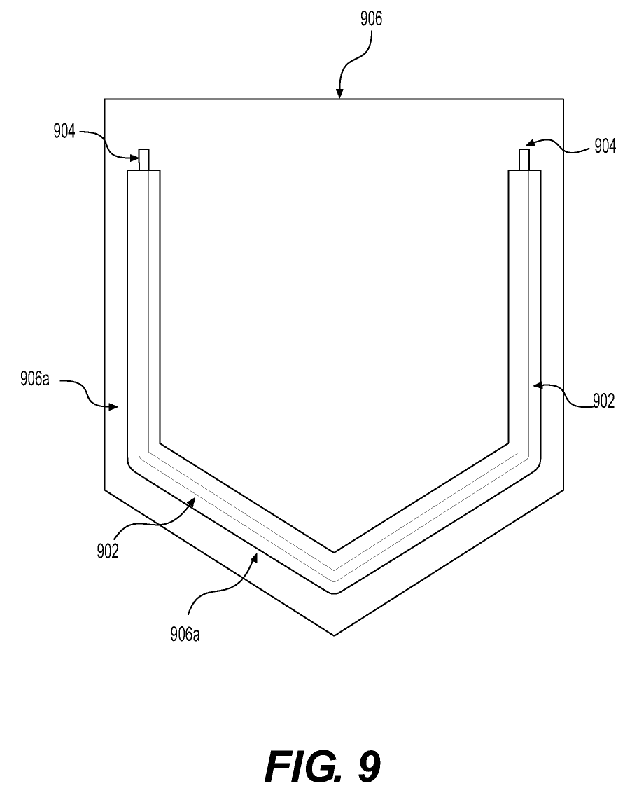

The exemplary coupling portions are shown in FIG. 9. For example, as schematically shown in FIG. 9, the adhesive composition 902 can be disposed between a portion of the plurality of struts 904 and leaflet 906. More specifically, the adhesive composition 902 can be disposed between a portion of the plurality of struts 904 and a portion of an edge portion of the leaflets 906a.

Yet, in other aspects, the adhesive composition can comprise a composition comprising a second polymer, an oligomer, a monomer, or a combination thereof. In certain aspects, the second polymer material and the first polymer material comprise the same polymer composition. Yet, in other aspects, the second polymer can comprise a polymer composition that is different from the first polymer. However, it is understood that in such aspects, the first and the second polymers are substantially compatible to form a seamless coupling portion. In yet still further aspects, while the polymer composition of the first and the second polymer can be the same, these polymers can still differ in molecular weight, different branching structures, and the like. In still further aspects, the molecular weight of the polymer composition and degree and type of branching can be tuned to obtain the desired viscosity and solubility characteristics of the adhesive composition.

It is understood that in aspects where the adhesive composition comprises an oligomer, a monomer, or a combination thereof, these oligomers and monomers can represent the same or different structural units of the first polymer and/or second polymer. For example, the oligomers and monomers in the adhesive composition can be different from any of the structural units of the first polymer. Yet, in other aspects, the oligomers and monomers of the adhesive composition can be represented by at least some of the backbone units that form the first polymer and/or second polymer. In still further aspects, in addition to the oligomers and monomers of the polymers disclosed above, the composition also can comprise oligomers and monomers of acrylates, epoxy, vinyl, or any combination thereof. Yet in still further aspects, if the adhesive composition comprises oligomers and monomers, it is understood that such a composition can be polymerized after the application to form the coupling portion. The polymerization can be done by any known in the art methods. For example, it can be polymerized by irradiation (e.g., heat, UV-radiation, IR-radiation, and the like).

In some exemplary and unlimiting aspects, when the first polymer and the second polymer have the same polymer composition, the coupling body forms a unibody with the one or more leaflets. In such exemplary and unlimiting aspects, the at least a portion of the plurality of struts is embedded into the adhesive composition, and the adhesive composition non-distinguishably expands into the leaflets. In such aspects, the coupling portion seamlessly extends into the edge portion of the leaflet, with substantially no rough edges formed at any point of the coupling. In still further aspects, the coupling portion can form an edge between the coupling portion and the at least a portion of the leaflet edge. In still further aspects, the adhesive composition substantially diffuses into the desired portion of the edge portion of the leaflet. Yet, in still further aspects, the adhesive composition can diffuse in a portion of the leaflet that extends beyond the edge portion of the leaflet.

In still further aspects, the coupling portion can have the same thickness as the original edge portion of the leaflet. Yet, in other aspects, the adhesive composition can form a reinforcing portion. In such aspects, the reinforcing portion can be formed by an additional layer of the adhesive that results in an increased thickness around the edge portion of the leaflets. Such a reinforcing portion further protects the leaflets from tearing up or any other mechanical damage. In still further aspects, the coupling portion can have a thickness of about 1 µm to about 500 µm, including exemplary values of about 2 µm, about 5 µm, about 10 µm, about 20 µm, about 30 µm, about 40 µm, about 50 µm, about 60 µm, about 70 µm, about 80 µm, about 90 µm, about 100 µm, about 150 µm, about 200 µm, about 250 µm, about 300 µm, about 350 µm, about 400 µm, and about 450 µm. It is understood that the thickness can have any value that falls between any two foregoing values or fall in a range that is formed by any two foregoing values. For example, and without limitations, the thickness can be about 10 µm to about 500 µm, about 50 µm to about 500 µm, about 100 µm to about 500 µm, about 200 µm to about 500 µm, about 300 µm to about 500 µm, about 400 µm to about 500 µm, about 1 µm to about 400 µm, about 1 µm to about 300 µm, about 1 µm to about 200 µm, about 1 µm to about 100 µm, about 1 µm to about 50 µm, and so on.

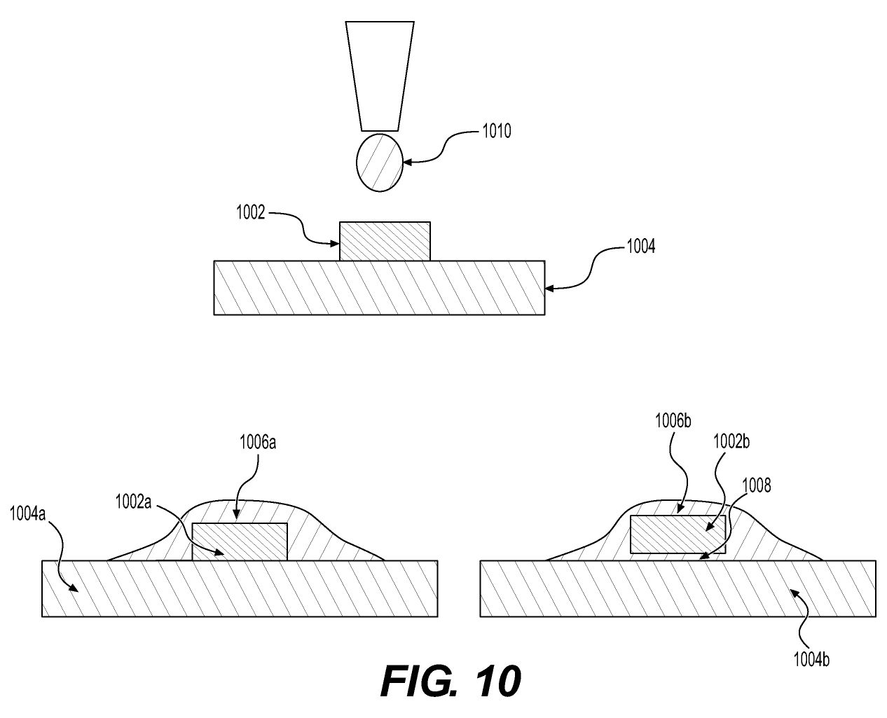

In still further aspects, the coupling portion comprises substantially no gap between the at least a portion of an edge portion of the one or more leaflets and the at least a portion of the plurality of struts. Yet, in other aspects, the coupling portion comprises no gap between the at least a portion of the edge portion of the leaflets and the at least a portion of the plurality of struts. An exemplary and unlimiting aspect of such an arrangement can be seen in FIG. 10. The portion of the plurality of struts 1002a is positioned adjacent to the portion of the leaflet 1004a, and the adhesive composition 1006a is disposed such that substantially no gap is formed between the leaflet and the frame. Yet in other aspects, the coupling portion can comprise a gap 1008 between at least a portion of an edge portion of the one or more leaflets 1004b and the at least a portion of the plurality of struts 1002b such that the gap 1008 is filled with the adhesive composition 1006b.

In still further aspects, if desired, the implantable prosthetic valve can also comprise a skirt. In certain aspects, the skirt is positioned on the outside of the frame, similarly to the skirt 16 in the conventional sutured valve (FIG. 1). The skirt can be formed from a textile material or silicon, depending on the desired application. In certain aspects, the skirt can be attached to the sutures. Yet, in other aspects, the skirt can be formed suturelessly. For example, and without limitations, the skirt can be formed by dipping the frame into a liquid silicon. In aspects where the outer skirt is present when crimped, frame 12 has a reduced diameter region extending along a portion of the frame adjacent to the inflow end of the frame that generally corresponds to the region of the frame covered by the outer skirt 16. Yet, in other aspects, the skirt can be formed suturelessly by using any of the disclosed herein first and/or second polymers and attaching such a skirt by the disclosed methods to at least a portion of the plurality of struts and at least a portion of the leaflets. In some aspects, the reduced diameter region is reduced compared to the diameter of the upper portion of the frame (which is not covered by the outer skirt) such that the outer skirt 16 does not increase the overall crimp profile of the prosthetic valve.

### METHODS

The present disclosure also provides for a method of forming an implantable prosthetic valve comprising: a) providing an annular frame having an inner surface and an outer surface wherein the frame has an inflow end and an outflow end, and a central longitudinal axis extending from the inflow end to the outflow end; wherein the annular frame comprises a plurality of struts; b) aligning at least a portion of an edge portion of one or more leaflets with a portion of the plurality of struts; and dispersing an adhesive composition to form an adhesive composition configured to couple the portion of the plurality of struts with the at least a portion of the edge portion of the one or more leaflets to form a sutureless coupling portion between the one or more leaflets and the plurality of struts of the annular frame, such that the coupling portion encapsulates the at least a portion of the plurality of struts and seamlessly extends into the at least a portion of an edge portion of the one or more leaflets.

In still further aspects, the one or more leaflets can comprise a first polymer. Any of the disclosed above first polymers can be utilized. In still further aspects, the adhesive composition can comprise a composition comprising a second polymer, an oligomer, a monomer, or a combination thereof and optionally a solvent. The second polymer, oligomer, and/or monomer, can comprise any of the disclosed above compounds. In some aspects, the adhesive composition comprises the solvent present in the adhesive composition. In such aspects, the solvent can comprise any known in the art solvents that can at least partially dissolve the first polymer and/or the second polymer and/or oligomer and/or monomer. In such exemplary and unlimiting aspects, the solvent can comprise dimethylacetamide (DMAc), dimethylformamide (DMF), tetrahydrofuran (THF), acetone, isopropyl alcohol (IPA), methyl ethyl ketone (MEK), butyl acetate, and the like, or any combination thereof.

In still further aspects, the adhesive composition can be substantially dissolved in the solvent and dispersed as a solution at at least a portion of the plurality of struts and at least a portion of the edge portion of the one or more leaflets. After dispersing the solution in the desired concentration and desired viscosity, in some aspects, some of the portion of the edge portion of the one or more leaflets can get slightly dissolved (or melted). In such aspects, the adhesive composition and the dissolved portion of the leaflet are substantially mixed and seamlessly connected upon the removal of the solvent. The solvent can be removed by any known in the art methods, for example, drying at room temperature and atmospheric pressure, in a vacuum, or at elevated temperature. In still further aspects, the solvent is removed at conditions effective to provide the desired coupling portion. It is understood that conditions are controlled to ensure that the leaflets and coupling portions do not lose the desired mechanical and chemical properties. It is further understood that the coupling portion formed by the disclosed herein methods forms a unibody with the one or more leaflets.

Some exemplary method steps are shown in FIG. 10. For example, the first polymer solution 1010 comprising the first polymer, and the solvent is dispersed on the metal strut 1002 and the polymer leaflet 1004 to encapsulate the strut and incorporate it into the leaflets. As discussed above, various combinations of the coupling portion can be formed. In some aspects, the coupling portion comprises substantially no gap between the strut and the leaflet. While in other aspects, a gap 1008 can also be formed between the strut and the leaflet.

In other aspects, the adhesive composition can comprise no solvent. In such exemplary aspects, the adhesive composition can be melted to form a melt solution and applied to the portions of the plurality of struts and the leaflets. In such aspects, the edge portion of the leaflet can also be melted to seamlessly incorporate the strut within the coupling portion and to form a unibody between the applied adhesive composition and the leaflets.

In still further aspects and as disclosed herein, the adhesive composition can comprise oligomers or monomers. They can be present in the solvent or without the solvent, as disclosed above. In certain aspects, the oligomers and/or monomers can be dispersed on the aligned struts and leaflets and then polymerized by applying radiation. In certain aspects, the radiation can be thermal UV-light or IR-light radiation. In yet still further aspects, if needed, the adhesive composition can comprise crosslinking materials or polymerization initiators that would allow the quick and efficient formation of the coupling portion. The specific irradiation conditions will be the conditions effective to provide the desired coupling portion.

In still further aspects, the adhesive composition, when applied, exhibits a viscosity of about 5 mPa·s to about 100,000 mPa·s, including exemplary values of about 10 mPa·s, about 20 mPa·s, about 50 mPa·s, about 100 mPa·s, about 250 mPa·s, about 500 mPa·s, about 750 mPa·s, about 1,000 mPa·s, about 5,000 mPa·s, about 10,000 mPa·s, about 15,000 mPa·s, about 20,000 mPa·s, about 25,000 mPa·s, about 30,000 mPa·s, about 35,000 mPa·s, about 40,000 mPa·s, about 45,000 mPa·s, about 50,000 mPa·s, about 55,000 mPa·s, about 60,000 mPa·s, about 65,000 mPa·s, about 70,000 mPa·s, about 75,000 mPa·s, about 80,000 mPa·s, about 85,000 mPa·s, about 90,000 mPa·s, and about 95,000 mPa·s. It is understood that the viscosity can have any value that falls between any two foregoing values or fall in a range that is formed by any two foregoing values. For example, and without limitations, the viscosity can be about 10 mPa·s to about 100,000 mPa·s, 50 mPa·s to about 100,000 mPa·s, 100 mPa·s to about 100,000 mPa·s, 500 mPa·s to about 100,000 mPa·s, 1,000 mPa·s to about 100,000 mPa·s, 10,000 mPa·s to about 100,000 mPa·s, 50,000 mPa·s to about 100,000 mPa·s, 5 mPa·s to about 50,000 mPa·s, 5 mPa·s to about 10,000 mPa·s, 5 mPa·s to about 1,000 mPa·s, 5 mPa·s to about 500 mPa·s, 5 mPa·s to about 100 mPa·s, 5 mPa·s to about 50 mPa·s, and so on.

In still further aspects, the adhesive composition exhibits a surface tension of about 20 mJ/m² to about 60 mJ/m², including exemplary values of about 25 mJ/m²,about 30 mJ/m²,about 35 mJ/m²,about 40 mJ/m²,about 45 mJ/m²,about 50 mJ/m², andabout 55 mJ/m². It is understood that the surface tension can have any value that falls between any two foregoing values or fall in a range that is formed by any two foregoing values. For example, and without limitations, the surface tension can be about 20 mJ/m² to about 50 mJ/m², about 20 mJ/m² to about 40 mJ/m², about 20 mJ/m² to about 30 mJ/m², about 30 mJ/m² to about 60 mJ/m², about 40 mJ/m² to about 60 mJ/m², about 50 mJ/m² to about 60 mJ/m², and so on.

In still further aspects, at least a portion of the plurality of struts is pretreated prior to the step of providing. In such aspects, the pretreatment can comprise any method step known in the art and suitable for the desired application. In certain aspects, the pretreatment can comprise plasma treatment, sterilization, electrolytic treatment, or any combination thereof. In still further aspects, the sterilization can comprise e-beam sterilization (if applicable), ethylene oxide gas sterilization, alcohol treatment, corona treatment, and the like. In yet other aspects, if desired, adhesion promoters or other materials that provide the desired properties to the frame can also be deposited. Such depositions can be done through an electrolytic deposition, a vapor deposition (chemical vapor deposition, atomic layer deposition, and the like), or any combination thereof.

In still further aspects, disclosed herein are methods that use the annular frame as a mold to form a unibody of leaflets and struts. In such exemplary and unlimiting aspects, the method can comprise providing an annular frame having an inner surface and an outer surface wherein the frame has an inflow end and an outflow end, and a central longitudinal axis extending from the inflow end to the outflow end; wherein the annular frame comprises a plurality of struts; and forming a polymeric valvular structure comprising one or more leaflets within the inner surface of the annular frame such that at least a portion of an edge portion of one or more leaflets form a unibody with at least a portion of the plurality of struts. In such aspects, the polymeric valvular structure is molded from a polymer composition within the inner surface of the annular frame. In some aspects, this polymer composition can comprise any of the disclosed above first polymer materials. In yet still further aspects, the polymer composition comprises a thermoplastic polyurethane.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. It is thus understood that the disclosure is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described disclosure nor the claims which follow. We, therefore, claim as our disclosure all that comes within the scope and spirit of these claims.

### EXEMPLARY ASPECTS

Example 1. An implantable prosthetic valve comprising: an annular frame having an inner surface and an outer surface wherein the frame has an inflow end and an outflow end, and a central longitudinal axis extending from the inflow end to the outflow end, wherein the annular frame comprises a plurality of struts; a valvular structure comprising one or more leaflets, each having an inner surface and an outer surface and positioned at the inner surface of the annular frame; wherein at least a portion of an edge portion of the one or more leaflets is suturelessly attached to at least a portion of the plurality of struts of the annular frame with an adhesive composition to form a coupling portion, wherein the coupling portion encapsulates the at least a portion of the plurality of struts and seamlessly extends into the at least a portion of an edge portion of the one or more leaflets.

Example 2. The implantable prosthetic valve of any one of examples herein, particularly example 1, wherein the annular frame comprises an expandable material.

Example 3. The implantable prosthetic valve of any one of examples herein, particularly example 2, wherein the expandable material comprises one or more of: a) a metal or metal alloys comprising stainless steel, cobalt-chromium alloy, cobalt-chromium-tungsten-nickel alloy, nickel-cobalt-chromium alloy, nickel-cobalt-chromium-molybdenum alloy, nitinol, or a combination thereof; and/or b) a polymer comprising polyether ether ketone (PEEK), polytetrafluoroethylene (PTFE), ultra-high-molecular-weight polyethylene (UHMWPE), polyimide, polysulfone (PSU), polyphenylsulfone (PPSU) (filled or unfilled), or any combination thereof.

Example 4. The implantable prosthetic valve of any one of examples herein, particularly examples 1–3, wherein the one or more leaflets comprise a first polymer material.

Example 5. The implantable prosthetic valve of any one of examples herein, particularly example 4, wherein the first polymer material comprises a polyurethane-based composition, polyurea composition, polytetrafluoroethylene (PTFE), silicon styrene isoprene butadiene (SIBS) block copolymers, or any combination thereof.

Example 6. The polymer material in any one of examples herein, particularly example 5, wherein the polyurethane-based composition comprises one or more of poly(ether urethanes), poly(ester urethanes), poly(carbonate urethanes), poly(siloxane urethanes), polyurethane-polyester composite, polyurethane-polyamide composite, polyurethane-glass fiber composite, polyurethane-carbon fiber composite, or any combination thereof.

Example 7. The implantable prosthetic valve of any one of examples herein, particularly examples 1–6, wherein the adhesive composition comprises a composition comprising a second polymer, an oligomer, a monomer, or a combination thereof.

Example 8. The implantable prosthetic valve of any one of examples herein, particularly example 7, wherein the first polymer material and the second polymer material are the same.

Example 9. The implantable prosthetic valve of any one of examples herein, particularly example 7, wherein the first polymer material and the second polymer are different.

Example 10. The implantable prosthetic valve of any one of examples herein, particularly examples 1–9, wherein the coupling portion forms a unibody with the one or more leaflets.

Example 11. The implantable prosthetic valve of any one of examples herein, particularly examples 1–10, wherein the coupling portion extends into the at least a portion of the edge portion of the one or more leaflets to form a reinforcing portion.

Example 12. The implantable prosthetic valve of any one of examples herein, particularly examples 1–11, wherein the coupling portion comprises no gap between the at least a portion of the edge portion of the one or more leaflets and the at least a portion of the plurality of struts.

Example 13. The implantable prosthetic valve of any one of examples herein, particularly examples 1–11, wherein the coupling portion comprises a gap between at least a portion of an edge portion of the one or more leaflets and the at least a portion of the plurality of struts such that the gap is filled with the adhesive composition.

Example 14. The implantable prosthetic valve of any one of examples herein, particularly examples 1–13, wherein the adhesive composition comprises a second polymer, oligomer, monomer, and optionally a solvent.

Example 15. The implantable prosthetic valve of any one of examples herein, particularly examples 1–13, wherein the adhesive composition comprises an oligomer, a monomer, or any composition thereof.

Example 16. The implantable prosthetic valve of any one of examples herein, particularly example 15, wherein the composition is radiation polymerized to form the coupling portion.

Example 17. The implantable prosthetic valve of any one of examples herein, particularly examples 1–16, wherein the coupling portion has a thickness of about 1 µm to about 500 µm.

Example 18. The implantable prosthetic valve of any one of examples herein, particularly examples 1–17, wherein the annular frame is pretreated prior to coupling with the at least a portion of the edge portion of the one or more leaflets.

Example 19. A method comprising: providing an annular frame having an inner surface and an outer surface wherein the frame has an inflow end and an outflow end, and a central longitudinal axis extending from the inflow end to the outflow end; wherein the annular frame comprises a plurality of struts; aligning at least a portion of an edge portion of one or more leaflets with a portion of the plurality of struts; and dispersing an adhesive composition to couple the portion of the plurality of struts with the at least a portion of the edge portion of the one or more leaflets to form a sutureless coupling portion between the one or more leaflets and the plurality of struts of the annular frame, such that the coupling portion encapsulates the at least a portion of the plurality of struts and seamlessly extends into the at least a portion of an edge portion of the one or more leaflets.

Example 20. The method of any one of examples herein, particularly example 19, wherein the annular frame comprises an expandable material.

Example 21. The method of any one of examples herein, particularly example 20, wherein the expandable material comprises one or more of: a) a metal or metal alloys comprising stainless steel, cobalt-chromium alloy, cobalt-chromium-tungsten-nickel alloy, nickel-cobalt-chromium alloy, nickel-cobalt-chromium-molybdenum alloy, nitinol, or a combination thereof; and/or b) a polymer comprising polyether ether ketone (PEEK), polytetrafluoroethylene (PTFE), ultra-high-molecular-weight polyethylene (UHMWPE), polyimide, polysulfone (PSU), polyphenylsulfone (PPSU) (filled or unfilled), or any combination thereof.

Example 22. The method of any one of examples herein, particularly examples 19–21, wherein the one or more leaflets comprise a first polymer material.

Example 23. The method of any one of examples herein, particularly example 22, wherein the first polymer material comprises a polyurethane-based composition, polyurea composition, PTFE, silicon rubber, SIBS, or any combination thereof.

Example 24. The method of any one of examples herein, particularly example 23, wherein polyurethane-based composition comprises one or more of poly(ether urethanes), poly(ester urethanes), poly(carbonate urethanes), poly(siloxane urethanes), polyurethane-polyester composite, polyurethane-polyamide composite, polyurethane-glass fiber composite, polyurethane-carbon fiber composite, or any combination thereof.

Example 25. The method of any one of examples herein, particularly examples 19–24, wherein the adhesive composition comprises a composition comprising a second polymer, an oligomer, a monomer, or a combination thereof and optionally a solvent.

Example 26. The method of any one of examples herein, particularly example 25, wherein the composition comprises the second polymer that is the same or different from the first polymer material.

Example 27. The method of any one of examples herein, particularly examples 25–26, wherein the method comprises evaporating the solvent to form the coupling portion between the at least a portion of the plurality of struts and the at least a portion of the edge portion of the one or more leaflets.

Example 28. The method of any one of examples herein, particularly examples 26 or 27, wherein the second polymer is the same as the first polymer and the coupling portion forms a unibody with the one or more leaflets.

Example 29. The method of any one of examples herein, particularly example 25, wherein when the adhesive composition comprises oligomer and/or monomer, the method comprises polymerizing the adhesive composition to form the adhesive composition that couples the portion of the plurality of struts with the at least a portion of the edge portion of the one or more leaflets by irradiating the adhesive composition after disposing it.

Example 30. The method of any one of examples herein, particularly example 25, wherein when the adhesive composition comprises oligomer and/or monomer, wherein the method comprises polymerizing the adhesive composition to form the adhesive composition that couples the portion of the plurality of struts with the at least a portion of the edge portion of the one or more leaflets by heating the adhesive composition after disposing it.

Example 31. The method of any one of examples herein, particularly examples 29 or 30, wherein the irradiating comprises exposing the adhesive composition to UV-light, IR-light, or any combination thereof.

Example 32. The method of any one of examples herein, particularly examples 19–31, wherein the adhesive composition exhibits a viscosity of about 5 mPa·s to about 100,000 mPa·s.

Example 33. The method of any one of examples herein, particularly examples 19–32, wherein the adhesive composition exhibits a surface tension of about 20 to about 60 mJ/m².

Example 34. The method of any one of examples herein, particularly examples 25–33, wherein the oligomer and/or monomer comprises urethane, acrylate, epoxy, vinyl, or any combination thereof.

Example 35. The method of any one of examples herein, particularly examples 19–34, wherein the portion of the plurality of struts is pretreated prior to the step of providing.

Example 36. The method of any one of examples herein, particularly example 35, wherein pretreatment comprises plasma treatment, sterilization, electrolytic treatment, or any combination thereof.

Example 37. The method of any one of examples herein, particularly examples 19–36, wherein the coupling portion extends into the at least a portion of the edge portion of the one or more leaflets to form a reinforcement layer.

Example 38. The method of any examples herein, particularly examples 19–37, wherein the coupling portion comprises no gap between the at least a portion of an edge portion of the one or more leaflets and the at least a portion of the plurality of struts.

Example 39. The method of any one of examples herein, particularly examples 19–37, wherein the coupling portion comprises a gap between at least a portion of an edge portion of the one or more leaflets and the at least a portion of the plurality of struts such that the gap is filled with the adhesive composition.

Example 40. A method comprising: providing an annular frame having an inner surface and an outer surface wherein the frame has an inflow end and an outflow end, and a central longitudinal axis extending from the inflow end to the outflow end; wherein the annular frame comprises a plurality of struts; and forming a polymeric valvular structure comprising one or more leaflets within the inner surface of the annular frame such that at least a portion of an edge portion of one or more leaflets form a unibody with at least a portion of the plurality of struts.

Example 41. The method of any one of examples herein, particularly example 40, wherein the polymeric valvular structure is molded from a polymer composition within the inner surface of the annular frame.

Example 42. The method of any one of examples herein, particularly example 41, wherein the polymer composition comprises a thermoplastic polyurethane.

We claim:

1. An implantable prosthetic valve comprising:
an annular frame having an inner surface and an outer surface wherein the frame has an inflow end and an outflow end, and a central longitudinal axis extending from the inflow end to the outflow end, wherein the annular frame comprises a plurality of struts;
a valvular structure comprising one or more leaflets, each having an inner surface and an outer surface and positioned at the inner surface of the annular frame;
wherein at least a portion of an edge portion of the one or more leaflets is suturelessly attached to at least a portion of the plurality of struts of the annular frame with an adhesive composition to form a coupling portion, wherein the coupling portion encapsulates the at least a portion of the plurality of struts and seamlessly extends into the at least a portion of an edge portion of the one or more leaflets.

2. The implantable prosthetic valve of any one of claims claim 1, wherein the annular frame comprises an expandable material comprising one or more of:
(a) a metal or metal alloys comprising stainless steel, cobalt-chromium alloy, cobalt-chromium-tungsten-nickel alloy, nickel-cobalt-chromium alloy, nickel-cobalt-chromium-molybdenum alloy, nitinol, or a combination thereof; and/or
(b) a polymer comprising polyether ether ketone (PEEK), polytetrafluoroethylene (PTFE), ultra-high-molecular-weight polyethylene (UHMWPE), polyimide, polysulfone (PSU), polyphenylsulfone (PPSU) (filled or unfilled), or any combination thereof.

3. The implantable prosthetic valve of claim 1, wherein the one or more leaflets comprise a first polymer material.

4. The implantable prosthetic valve of claim 3, wherein the first polymer material comprises a polyurethane-based composition, polyurea composition, polytetrafluoroethylene (PTFE), silicon styrene isoprene butadiene (SIBS) block copolymers, or any combination thereof.

5. The polymer material in claim 4, wherein the polyurethane-based composition comprises one or more of poly(ether urethanes), poly(ester urethanes), poly(carbonate urethanes), poly(siloxane urethanes), polyurethane-polyester composite, polyurethane-polyamide composite, polyurethane-glass fiber composite, polyurethane-carbon fiber composite, or any combination thereof.

6. The implantable prosthetic valve of claim 1, wherein the adhesive composition comprises a composition comprising a second polymer, an oligomer, a monomer, or a combination thereof.

7. The implantable prosthetic valve of claim 1, wherein
the coupling portion forms a unibody with the one or more leaflets; and/or
the coupling portion extends into the at least a portion of the edge portion of the one or more leaflets to form a reinforcing portion.

8. The implantable prosthetic valve of claim 1, wherein
the coupling portion comprises no gap between the at least a portion of the edge portion of the one or more leaflets and the at least a portion of the plurality of struts; or
the coupling portion comprises a gap between at least a portion of an edge portion of the one or more leaflets and the at least a portion of the plurality of struts such that the gap is filled with the adhesive composition.

9. The implantable prosthetic valve of claim 1,
wherein the adhesive composition comprises a second polymer, oligomer, monomer, and, optionally, a solvent or
wherein the adhesive composition comprises an oligomer, a monomer, or any composition thereof.

10. The implantable prosthetic valve of claim 1, wherein the coupling portion has a thickness of about 1 µm to about 500 µm.

11. A method comprising:
providing an annular frame having an inner surface and an outer surface wherein the frame has an inflow end and an outflow end, and a central longitudinal axis extending from the inflow end to the outflow end; wherein the annular frame comprises a plurality of struts;
aligning at least a portion of an edge portion of one or more leaflets with a portion of the plurality of struts; and
dispersing an adhesive composition to couple the portion of the plurality of struts with the at least a portion of the edge portion of the one or more leaflets to form a sutureless coupling portion between the one or more leaflets and the plurality of struts of the annular frame, such that the coupling portion encapsulates the at least a portion of the plurality of struts and seamlessly extends into the at least a portion of an edge portion of the one or more leaflets.

12. The method of claim 11, wherein the annular frame comprises an expandable material comprising one or more of:
(a) a metal or metal alloys comprising stainless steel, cobalt-chromium alloy, cobalt-chromium-tungsten-nickel alloy, nickel-cobalt-chromium alloy, nickel-cobalt-chromium-molybdenum alloy, nitinol, or a combination thereof; and/or
(b) a polymer comprising polyether ether ketone (PEEK), polytetrafluoroethylene (PTFE), ultra-high-molecular-weight polyethylene (UHMWPE), polyimide, polysulfone (PSU), polyphenylsulfone (PPSU) (filled or unfilled), or any combination thereof.

13. The method of claim 11, wherein the one or more leaflets comprise a first polymer material comprising a polyurethane-based composition, polyurea composition, PTFE, silicon rubber, SIBS, or any combination thereof.

14. The method of claim 13, wherein polyurethane-based composition comprises one or more of poly(ether urethanes), poly(ester urethanes), poly(carbonate urethanes), poly(siloxane urethanes), polyurethane-polyester composite, polyurethane-polyamide composite, polyurethane-glass fiber composite, polyurethane-carbon fiber composite, or any combination thereof.

15. The method of claim 13, wherein the adhesive composition comprises a composition comprising a second polymer, an oligomer, a monomer, or a combination thereof and optionally a solvent, and wherein the second polymer that is the same or different from the first polymer material.

16. The method of claim 15, wherein the method comprises evaporating the solvent, if present, to form the coupling portion between the at least a portion of the plurality of struts and the at least a portion of the edge portion of the one or more leaflets.

17. The method of claim 15, wherein the second polymer is the same as the first polymer and the coupling portion forms a unibody with the one or more leaflets.

18. The method of claim 15, wherein
when the adhesive composition comprises oligomer and/or monomer, the method comprises polymerizing the adhesive composition to form the adhesive composition that couples the portion of the plurality of struts with the at least a portion of the edge portion of the one or more leaflets by irradiating the adhesive composition after disposing it; or
wherein when the adhesive composition comprises oligomer and/or monomer, wherein the method comprises polymerizing the adhesive composition to form the adhesive composition that couples the portion of the plurality of struts with the at least a portion of the edge portion of the one or more leaflets by heating the adhesive composition after disposing it.

19. The method of claim 11, wherein the adhesive composition exhibits a viscosity of about 5 mPa·s to about 100,000 mPa·s and/or
exhibits a surface tension of about 20 to about 60 mJ/m2.

20. The method of claim 15, wherein the oligomer and/or monomer comprises urethane, acrylate, epoxy, vinyl, or any combination thereof.

21. The method of claim 11, wherein the portion of the plurality of struts is pretreated prior to the step of providing, wherein pretreatment comprises plasma treatment, sterilization, electrolytic treatment, or any combination thereof.

22. The method of claim 11, wherein
the coupling portion extends into the at least a portion of the edge portion of the one or more leaflets to form a reinforcement layer; or
the coupling portion comprises no gap between the at least a portion of an edge portion of the one or more leaflets and the at least a portion of the plurality of struts; or
the coupling portion comprises a gap between at least a portion of an edge portion of the one or more leaflets and the at least a portion of the plurality of struts such that the gap is filled with the adhesive composition.

23. A method comprising:
providing an annular frame having an inner surface and an outer surface wherein the frame has an inflow end and an outflow end, and a central longitudinal axis extending from the inflow end to the outflow end; wherein the annular frame comprises a plurality of struts; and
forming a polymeric valvular structure comprising one or more leaflets within the inner surface of the annular frame such that at least a portion of an edge portion of one or more leaflets forms a unibody with at least a portion of the plurality of struts.

24. The method of claim 23, wherein the polymeric valvular structure is molded from a polymer composition within the inner surface of the annular frame, and wherein the polymer composition comprises a thermoplastic polyurethane.
